(19) United States
(12) Patent Application Publication
(10) Pub. No.: US 2013/0006379 A1
(43) Pub. Date: Jan. 3, 2013

(54) Talar-calcaneal sinus-canalis internal-fixation device

(76) Inventor: Paul Clint Jones, Post Falls, ID (US)

(21) Appl. No.: 13/135,226

(22) Filed: Jun. 29, 2011

(51) Int. Cl. A61F 2/42

(52) U.S. Cl. 623/21.18

(57) ABSTRACT

A sinus-canalis internal-fixation device is configured in a shape modeled after the anatomical form and dimensions of a sinus tarsi of an ankle-bone structure of a patient, which anatomically twists and curves and is surrounded by the anatomically irregular surfaces of the talus (ankle bone) and calcaneus (heel bone). The surfaces of the sinus-canalis internal-fixation device mirror the anatomically irregular surfaces of the talus (ankle bone) and calcaneus (heel bone) surrounding the sinus-canalis internal-fixation device. The sinus-canalis internal-fixation device comprises an anatomical shaft and anatomical superior, inferior, and posterior pegs connected to the top, bottom, and back end of the anatomical shaft, respectively. Further, if desired, the sinus-canalis internal-fixation device can be cannulated and/or comprise at least one groove, recess, opening, ridge, and/or hill integrated thereinto. The sinus-canalis internal-fixation device can:
a) Block the anterior, medial translation and internal, medial rotation of the talus on the calcaneus to obviate limitations in correcting abnormal foot mechanics,
b) Distribute the body weight of the patient over a maximum contact area between the sinus-canalis internal-fixation device and the talus (ankle bone) and calcaneus (heel bone),
c) Absorb the shocks caused by the body weight of the patient,
d) Create coupling-force affect to prevent superior and inferior togglings of the sinus-canalis internal-fixation device within the sinus tarsi to eliminates the problem of displacement and failure of the sinus-canalis internal-fixation device,
e) Correct an anatomically deformed alignment of the ankle-bone structure,
f) Maintain the ankle-bone structure in an anatomically correct alignment, and
g) Eliminate the need for having to verify the anatomically correct alignment of the ankle-bone structure with a fluoroscope, and thus eliminate the need for exposing the patient to radiation.

### BACKGROUND OF THE INVENTION

1. Field of the Invention

This invention is in the field of fixation of foot joint subluxation or dislocation deformities that impede and/or deteriorate optimal ambulatory mechanics. Particularly, the present invention relates to a talar-calcaneal sinus-canalis internal-fixation device having a shape modeled after the anatomical shape of a sinus tarsi of a patient, and having an anatomical superior peg, an anatomical inferior peg, and an anatomical posterior peg.

2. Description of the Prior Art

The untwisting and subsequent collapsing of the foot (exorotation) is caused by excessive motion between the talus (ankle bone) and the calcaneus (heel bone) of a foot. This excessive motion will eventually lead to anatomical poor-alignment of both proximal and distal joints surrounding the talus (ankle bone). The abnormal motion is due to obliteration or closure of the sinus (naturally occurring space) formed between the talus (ankle bone) and calcaneus (heel bone), and/or due to progressive, peri-articular subluxation or dislocation of the joints associated with these bones. This is commonly stated as being “double jointed”. In a foot, due to the cumulative affects of gravitational forces with each step, this results in progressive, increased dislocation of the peri-talar joints with tearing of surrounding joint capsules and tendons, and also results in arthritis.

A number of prior-art have been introduced to correct the deformity in the ankle-bone structure. U.S. Pat. No. 4,450,591, filed Dec. 10, 1981, to Mark J. Rappaport; U.S. Pat. No. 4,973,333, filed Aug. 10, 1988, to Richard Treharne; U.S. Pat. No. 5,007,930, filed Aug. 6, 1990, to Linneaus C. Dorman; U.S. Pat. No. 5,057,109, filed Mar. 7, 1990, to Sven Olerud; U.S. Pat. No. 5,084,050, filed Oct. 2, 1989, to Klaus Draenert; U.S. Pat. No. 5,207,712, filed May 7, 1992, to Michael Cohen; U.S. Pat. No. 5,300,076, filed Oct. 9, 1992, to Bertrand Leriche; U.S. Pat. No. 5,360,450, filed Mar. 9, 1993, to Sandro Giannini; U.S. Pat. No. 5,531,792, filed Jun. 14, 1994, to Donald R. Huene; U.S. Pat. No. 5,741,253, filed Oct. 29, 1992, to Gary Karlin Michelson; U.S. Pat. No. 5,766,253, filed Jan. 16, 1996, to Robert E. Brosnahan, III; U.S. Pat. No. 5,776,196, filed Mar. 5, 1996, to Matsuzaki, et al.; U.S. Pat. No. 5,785,710, filed Jun. 7, 1995, to Gary Karlin Michelson; U.S. Pat. No. 5,957,953, filed Feb. 16, 1996, to DiPoto, et al.; U.S. Pat. No. 6,053,920, filed Jun. 12, 1998, to Carlsson et al.; U.S. Pat. No. 6,136,032, filed Sep. 7, 1999, to Vilado Perice, et al.; U.S. Pat. No. 6,168,631, filed Aug. 29, 1997, to Mawell, et al.; U.S. Pat. No. 6,443,954, filed Apr. 24, 2001, to Bramlet et al.; U.S. Pat. No. 6,607,535, filed Feb. 4, 1999, to Kwan-Ho Chan; U.S. Pat. No. 7,033,398, filed Feb. 19, 2004, to Graham, Michael; U.S. Pub. No. 2005/0177165, filed Feb. 11, 2004, to Zang, Kerry; U.S. Pub. No. 2005/0177243, filed Feb. 1, 2005, to Lepow, Gary; U.S. Pub. No. 2008/0208349, filed Feb. 23, 2007, to Graser, Robert disclose a variety of inventions related to devices for correcting the deformity in the ankle-bone structure. The prior-art has failed to solve many problems associated with such internal-correction devices, as follows:

1) The prior art is configured in a geometric shape, which is not anatomically modeled after the anatomical shape of a sinus tarsi of a patient. Therefore, the geometrically shaped prior art (for example, U.S. Pat. No. 5,360,450; U.S. Pat. No. 6,136,032; U.S. Pat. No. 6,168,631; U.S. Pat. No. 7,033,398) cuts, grinds, wears, deforms, damages the talus (ankle bone), calcaneus (heel bone), surrounding tissues, ligaments, veins, arteries, and nerve systems when the bodyweight of a patient pounds on the prior art through the talus, calcaneus, surrounding tissues, ligaments, veins, arteries, and nerve systems at every step the patient makes. This leads to many problems of excruciating pain, the fracture and weakening of the talus and calcaneus, the deformity and damage of surrounding tissues, ligaments, veins, arteries, and nerve systems, and the failure of the prior-art implantation.

2) The prior art can not distribute the body weight of a patient over the entire circular surface of the prior art because the prior art has a circular surface, which can only create a minimal contact area with the anatomically irregular surfaces of the talus (ankle bone) and the calcaneus (heel bone) of the ankle-bone structure of a patient. Therefore, the circular-surface prior art (for example, U.S. Pat. No. 5,360,450; U.S. Pat. No. 6,136,032; U.S. Pat. No. 6,168,631; U.S. Pat. No. 7,033,398) cuts, grinds, wears, deforms, damages the talus (ankle bone), calcaneus (heel bone), surrounding tissues, ligaments, veins, arteries, and nerve systems when the bodyweight of the patient pounds on the prior art through the talus, calcaneus, surrounding tissues, ligaments, veins, arteries, and nerve systems at every step the patient makes. This leads to many problems of excruciating pain, the fracture and weakening of the talus and calcaneus, the deformity and damage of surrounding tissues, ligaments, veins, arteries, and nerve systems, and the failure of the prior-art implantation.

3) The prior art is configured in a geometric shape having exposed, sharp thread on the surface of the prior art. Therefore, the exposed-sharp-thread prior art (for example, U.S. Pat. No. 5,360,450; U.S. Pat. No. 6,136,032; U.S. Pat. No. 6,168,631; U.S. Pat. No: 7,033,398) cuts, grinds, wears, deforms, damages the talus (ankle bone), calcaneus (heel bone), surrounding tissues, ligaments, veins, arteries, and nerve systems when the bodyweight of the patient pounds on the prior art through the talus, calcaneus, surrounding tissues, ligaments, veins, arteries, and nerve systems at every step the patient makes. This leads to many problems of excruciating pain, the fracture and weakening of the talus and calcaneus, the deformity and damage of surrounding tissues, ligaments, veins, arteries, and nerve systems, and the failure of the prior-art implantation.

4) The prior art is configured in a geometric shape having circular cross-section, which can only create a minimal contact area with the talus (ankle bone) and the calcaneus (heel bone) of a foot of a patient. Therefore, the circular-cross-section prior art (for example, U.S. Pat. No. 5,360,450; U.S. Pat. No. 6,136,032; U.S. Pat. No. 6,168,631; U.S. Pat. No. 7,033,398) cuts, grinds, wears, deforms, damages the talus (ankle bone), calcaneus (heel bone), surrounding tissues, ligaments, veins, arteries, and nerve systems when the bodyweight of the patient pounds on the prior art through the talus, calcaneus, surrounding tissues, ligaments, veins, arteries, and nerve systems at every step the patient makes. This leads to many problems of excruciating pain, the fracture and weakening of the talus and calcaneus, the deformity and damage of surrounding tissues, ligaments, veins, arteries, and nerve systems, and the failure of the prior-art implantation.

5) The prior art does not offer any blocking pegs to block the anterior, medial translation and internal, medial rotation of the talus (ankle bone) on the calcaneus (heel bone) of the ankle-bone structure to obviate limitations in correcting abnormal foot mechanics. The prior art can only minimize the excessive, abnormal motion. This often results in the failure of the prior-art implantation.

6) The prior art does not offer any blocking pegs to create coupling-force affect to prevent superior and inferior togglings of the prior art within a sinus tarsi of a patient to eliminate the problem of displacement and failure of the prior art.

7) The prior art can not absorb the shocks caused by the body weight of a patient at every step the patient makes because the prior art does not offer any shaft or pegs, whose surfaces are modeled after the anatomically irregular surfaces of the talus (ankle bone), calcaneus (heel bone) of the ankle-bone structure of a patient to distribute the body weight of the patient over their entire anatomically irregular surfaces.

Therefore, there exists a continuing need for a new, improved, easy-to-operate, and safe device to correct the deformity in the ankle-bone structure. In this regard, the present invention fulfills this need.

### Unique Features and Functions

The present invention substantially departs from the conventional concepts and designs of the prior art, and in doing so provides a unique talar-calcaneal sinus-canalis internal-fixation device, having a shape modeled after the anatomical form and demensions of a sinus tarsi of a patient and having an anatomical superior peg, an anatomical inferior peg, and an anatomical posterior peg. The unique talar-calcaneal sinus-canalis internal-fixation device can:
a) Block the anterior, medial translation and internal, medial rotation of the talus on the calcaneus of the ankle-bone structure of the patient to obviate limitations in correcting abnormal foot mechanics,
b) Distribute the body weight of the patient over a maximum contact area between the sinus-canalis internal-fixation device and the talus (ankle bone) and calcaneus (heel bone),
c) Absorb the shocks caused by the body weight of the patient,
d) Create coupling-force affect to prevent superior and inferior togglings of the sinus-canalis internal-fixation device within the sinus tarsi of the patient to eliminates the problem of displacement and failure of the sinus-canalis internal-fixation device,
e) Correct an anatomically deformed alignment of the ankle-bone structure,
f) Maintain the ankle-bone structure in an anatomically correct alignment, and
g) Eliminate the need for having to verify the anatomically correct alignment of the ankle-bone structure with a fluoroscope, and thus eliminate the need for exposing the patient to radiation.

### Objects and Advantages of the Invention

1) One object of the invention is that the sinus-canalis internal-fixation device distributes the bodyweight of a patient over its entire surfaces, by being modeled after the anatomical form and dimensions of a sinus canalis, which anatomically twists and curves and is surrounded by the anatomically irregular surfaces of the talus (ankle bone) and calcaneus (heel bone).

2) Another object of the invention is that the sinus-canalis internal-fixation device absorbs shocks, caused by the bodyweight of a patient at every step the patient makes, by structuring its entire surfaces to mirror the anatomically irregular surfaces of the talus (ankle bone) and calcaneus (heel bone) surrounding the sinus-canalis internal-fixation device such that the sinus-canalis internal-fixation device distributes the bodyweight of the patient over its entire surfaces.

3) Another object of the invention is that the sinus-canalis internal-fixation device offers unprecedented fit, by being modeled after the anatomical form and dimensions of a sinus canalis such that the entire surfaces of the sinus-canalis internal-fixation device mirror the entire surrounding surfaces of the talus (ankle bone) and calcaneus (heel bone).

4) Another object of the invention is that the sinus-canalis internal-fixation device offers unprecedented comfort, by being modeled after the anatomical form and dimensions of a sinus canalis such that the entire surfaces of the sinus-canalis internal-fixation device mirror the entire surrounding surfaces of the talus (ankle bone) and calcaneus (heel bone), and are free of prior-art circular cross-section and sharp-edge threads.

5) Another object of the invention is that the sinus-canalis internal-fixation device utilizes its opposite-coupling-force blocking pegs to block excessive, exorotational end-range-of-motion (unraveling) of the subtalar joint and to block abnormal subluxation or dislocation between the talus (ankle bone) and calcaneus (heel bone) while maintaining normal motion and alignment.

6) Another object of the invention is that the left and right forward-moving-only grooves of the sinus-canalis internal-fixation device function similarly as an arrowhead: a) Pushing tissues outwards when advancing to allow the sinus-canalis internal fixation device to be inserted easily into the sinus canalis; b) Pushing tissues inwards when backing up to prevent the displacement of the sinus-canalis internal-fixation device; and c) Securing the sinus-canalis internal-fixation device.

7) Another object of the invention is to obviate limitations in correcting abnormal foot mechanics.

8) Another object of the invention is to ensure proper foot motion, by stabilizing the end-range-of-motion between the talus (ankle bone) and calcaneus (heel bone).

9) Another object of the invention is to ensure that both the medial and lateral aspects of the talus (ankle bone) and calcaneus (heel bone) are stabilized.

10) A further object of this invention is to correct poor-alignment, both proximally and distally, of the joints surrounding the talus (ankle bone) and calcaneus (heel bone).

11) A further object of the invention is to provide a sinus-canalis internal-fixation device, that will not wear or deform the talus (ankle bone) and calcaneus (heel bone) over time.

12) A further object of the invention is to provide a sinus-canalis internal-fixation device, that will not wear or deform over time and, thus, fail.

13) A further object of the invention is to provide a sinus-canalis internal-fixation device, that will remain in place without a separate implant-anchoring procedure.

14) Another further object of the invention is to provide a method of correctly positioning a sinus-canalis internal-fixation device in the sinus canalis between the talus (ankle bone) and calcaneus (heel bone) without having to verify the correct position with a fluoroscope and, thus, without exposing a patient to radiation.

15) Another further object of the invention is to provide a minimally invasive method for implanting a sinus-canalis internal-fixation device.

16) Another further object of the invention is to provide a sinus-canalis internal-fixation device without requiring post-operative casting of the extremity.

17) Another further object of the invention is to provide a sinus-canalis internal-fixation device, which allows early post-operative ambulation.

Other objects and advantages of the present invention will become apparent from the following description of the sinus-canalis internal-fixation device taken in conjunction with the accompanying drawings.

### BRIEF DESCRIPTION OF THE DRAWINGS

FIG. 1 illustrates the top view of the bone structure of a human foot with a sinus-canalis internal-fixation device in a predetermined location, displaying the rotational axis of the subtalar joint.

FIG. 2 illustrates the side view of the bone structure of the foot with the sinus-canalis internal-fixation device in a predetermined location, displaying the rotational axis of the subtalar joint.

FIG. 3 illustrates the perspective view of the sinus-canalis internal-fixation device, whose superior and inferior pegs curve sidewards.

FIG. 4 illustrates the front view of the sinus-canalis internal-fixation device, whose superior and inferior pegs curve sidewards.

FIG. 5 illustrates the front view of the sinus-canalis internal-fixation device, whose superior and inferior pegs twist.

FIG. 6 illustrates the side view of the sinus-canalis internal-fixation device, whose superior, inferior, and posterior pegs curves upwards, curves downwards, and extends backwards, respectively.

FIG. 7 illustrates a broken-away view taken in the direction of arrow “7” in FIG. 2, of the left foot with the sinus-canalis internal-fixation device in a predetermined location.

FIGS. 8 and 9 illustrate the front view of the sinus-canalis internal-fixation device and the opposite motional directions of its superior and inferior pegs.

FIGS. 10 and 11 illustrate the side view of the sinus-canalis internal-fixation device and the opposite motional directions of the superior, inferior, and posterior pegs of the sinus-canalis internal-fixation device.

FIG. 12 illustrates the front view of the sinus-canalis internal-fixation device having an insertion recess for an insertion device to be inserted thereinto.

FIG. 13 illustrates the cross-sectional view of the sinus-canalis internal-fixation device.

FIGS. 14 and 15 illustrate the front and side views of the sinus-canalis internal-fixation device, which distributes the bodyweight of a patient over its entire anatomical surfaces.

FIG. 16 illustrates the side view of the sinus-canalis internal-fixation device having left and right forward-moving-only grooves disposed in an arrowhead-like disposition.

FIGS. 17, 18, 19, 20, and 21 illustrate variations of the sinus-canalis internal-fixation device.

FIGS. 22 and 23 illustrate other variations of the sinus-canalis internal-fixation device.

### SUMMARY OF THE INVENTION

The present invention accomplishes and offers the foregoing objects and advantages, respectively. The sinus-canalis internal-fixation device maintains the subtalar joint in anatomically correct alignment (maximal joint-surface contact), which allows the normal physiological motion to occur while eliminating the tendency for excessive, exorotational end-range-of-motion.

The sinus-canalis internal-fixation device is configured in a shape modeled after the anatomical form and dimensions of a sinus tarsi, which anatomically twists and curves and is surrounded by the anatomically irregular surfaces of the talus (ankle bone) and calcaneus (heel bone). For example, the sinus-canalis internal-fixation device can be modeled after a three-dimensional CAD scan of a sinus canalis of a patient such that the sinus-canalis internal-fixation device has the anatomical form and dimensions of the sinus canalis (which anatomically twists and curves) and has anatomically irregular surfaces (which mirror the anatomically irregular surfaces of the talus (ankle bone) and calcaneus (heel bone) surrounding the sinus-canalis internal-fixation device).

The sinus-canalis internal-fixation device, having an anatomical superior peg, an anatomical inferior peg, and an anatomical posterior peg, can:
a) Block the anterior, medial translation and internal, medial rotation of the talus on the calcaneus to obviate limitations in correcting abnormal foot mechanics,
b) Distribute the body weight of the patient over a maximum contact area between the sinus-canalis internal-fixation device and the talus (ankle bone) and calcaneus (heel bone),
c) Absorb the shocks caused by the body weight of the patient,
d) Create coupling-force affect to prevent superior and inferior togglings of the sinus-canalis internal-fixation device within the sinus tarsi to eliminates the problem of displacement and failure of the sinus-canalis internal-fixation device,
e) Correct an anatomically deformed alignment of the ankle-bone structure,
f) Maintain the ankle-bone structure in an anatomically correct alignment, and
g) Eliminate the need for having to verify the anatomically correct alignment of the ankle-bone structure with a fluoroscope, and thus eliminate the need for exposing the patient to radiation.

Further, if desired, the sinus-canalis internal-fixation device can be cannulated, can have smooth or grainy texture, and/or can comprise at least one recess, opening, ridge, hill, the like, the equivalent, or a combination of at least two of the above (e.g., groove, channel, canal, hole, through-hole, pore, etc.) integrated thereinto at predetermined locations and orientations, such that they:
a) Allow the sinus-canalis internal-fixation device to advance easily into a sinus canalis, and
b) Secure the sinus-canalis internal-fixation device in a sinus canalis.

### PREFERRED EMBODIMENT

### Structure

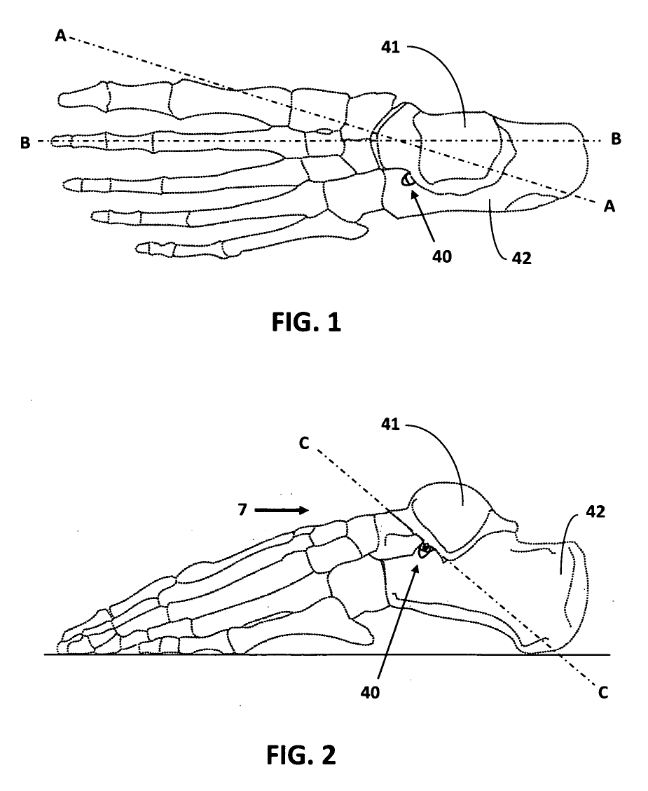

FIG. 1 illustrates the top view of the bone structure of a human foot with a sinus-canalis internal-fixation device 40 disposed in the sinus canalis of the foot. Sinus-canalis internal-fixation device 40 selectively blocks end-range-of-motion of the subtalar joint of the foot by exerting impeding oppositions to the translation and rotation of the surfaces of the subtalar joint. The subtalar joint is the articulation between a talus 41, superiorly, and a calcaneus 42, inferiorly. An axis A-A illustrates the subtalar-joint motion, which is approximately 16 degrees measured from a midline axis B-B of the foot.

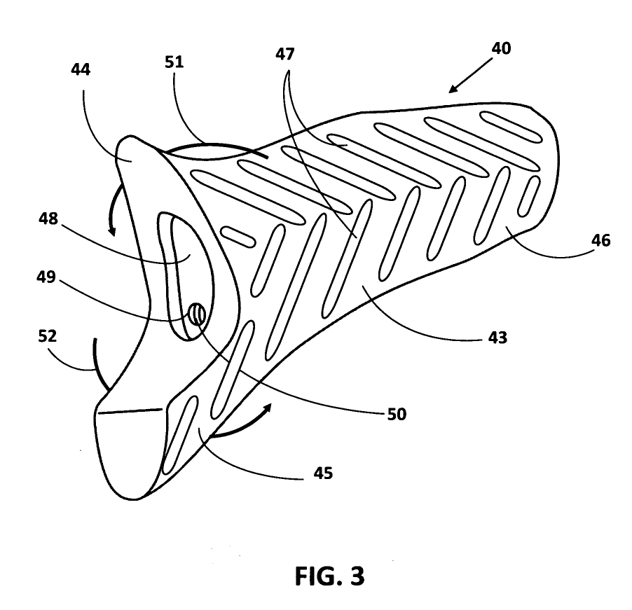

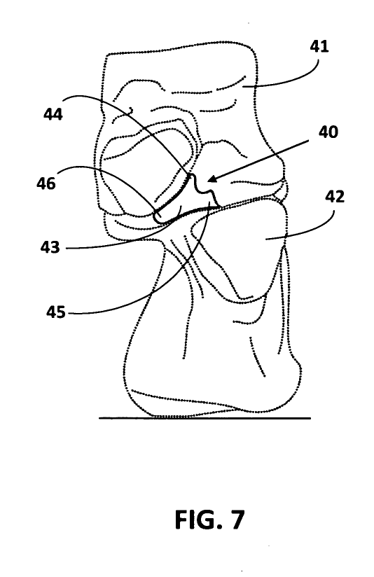

FIG. 2 illustrates the side view of the bone structure of the foot with sinus-canalis internal-fixation device 40 disposed in the sinus canalis of the foot. An axis C-C illustrates the subtalar-joint motion, which is approximately 42 degrees measured from a horizontal plane. FIG. 2 further illustrates a view direction “7” for FIG. 7 below.

The sinus canalis is posterior to (behind) the talocalcaneonavicular joint, which comprises
the middle and anterior calcaneal facet of talus 41 and
the middle and anterior talar facet of calcaneus 42.

The sinus canalis is anterior to (in front of) the subtalar joint, which comprises
the posterior calcaneal facet of talus 41 and
the posterior talar facet of calcaneus 42.

FIG. 3 illustrates the perspective view of sinus-canalis internal-fixation device 40. Sinus-canalis internal fixation device 40 comprises an anatomical shaft 43, an anatomical superior peg 44, an anatomical inferior peg 45, and an anatomical posterior peg 46. Sinus-canalis internal fixation device 40 has left and right forward-moving-only grooves 47. Anatomical shaft 43 has an insertion recess 48, a guiding cannula 49, and an extraction thread 50.

Sinus-canalis internal-fixation device 40 is modeled after a three-dimensional CAD scan of the sinus canalis of a patient such that, partially or entirely, sinus-canalis internal-fixation device 40 has the anatomical form and dimensions of the sinus canalis (which anatomically twists and curves) and has anatomically irregular surfaces (which mirror the anatomically irregular surfaces of talus 41 and calcaneus 42 surrounding the sinus canalis).

As a result, anatomical shaft 43 has an anatomically tapering, twisting, and curving body with anatomically elliptical cross-section. Anatomical superior peg 44 has a pyramid shape and is integrated into the top area of the front end of anatomical shaft 43. Anatomical inferior peg 45 has a sectional-elliptical-cylinder shape and is integrated into the bottom area of the front end of anatomical shaft 43. Anatomical posterior peg 46 has an elliptical-cylinder shape and is integrated into the back end of anatomical shaft 43. Left and right forward-moving-only grooves 47 each have an elliptical or ovoid shape and are integrated into the left and right sides of sinus-canalis internal-fixation device 40, respectively, at predetermined locations and orientations, such that both left and right forward-moving-only grooves 47 point toward left and right longitudinal axes of the left and right sides of sinus-canalis internal-fixation device 40, respectively. Insertion recess 48 has an elliptical or ovoid shape and is integrated into the front end of anatomical shaft 43. Guiding cannula 49 has a circular or elliptical perimeter and traverses the entire combined lengths of anatomical shaft 43 and anatomical posterior peg 46. Extraction thread 50 has the shape of a cylindrical or conical vortex and is integrated into the front end of guiding cannula 49.

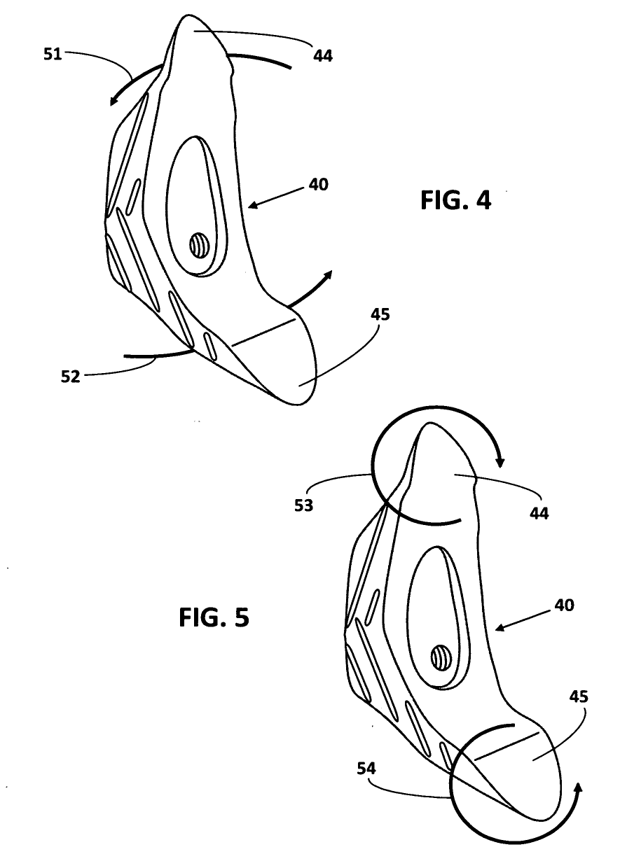

FIGS. 3 and 4 illustrate the perspective and front views of sinus-canalis internal-fixation device 40. As a result of being modeled after the anatomical form of the sinus canalis, superior and inferior pegs 44 and 45 curve sidewards in the directions of arrows 51 and 52, respectively.

FIG. 5 illustrates the front view of sinus-canalis internal-fixation device 40. As a result of being modeled after the anatomical form of the sinus canalis, superior and inferior pegs 44 and 45 also twist in the directions of arrows 53 and 54, respectively.

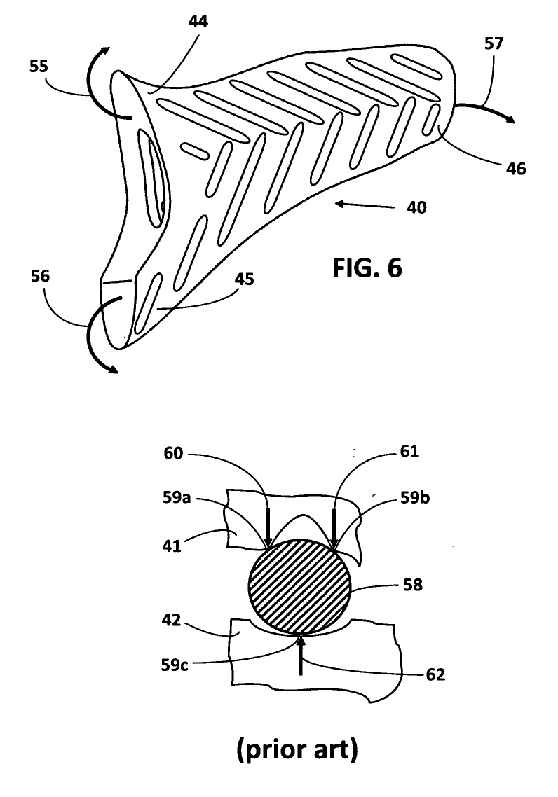

FIG. 6 illustrates the side view of sinus-canalis internal-fixation device 40. As a result of being modeled after the anatomical form of the sinus canalis, superior peg 44 also curves upwards in the direction of arrow 55, inferior peg 45 also curves downwards in the direction of arrow 56, and posterior peg 46 curves downwards in the direction of arrow 57.

### Material

Sinus-canalis internal-fixation device 40 can be made entirely from a single material, which, for example, can comprise a medical-grade polymer suitable for the insertion in the body in that it is substantially inert with respect to chemical reactions present in the body and is unlikely to result in adverse reactions, infections, adverse immunologic reactions such as allergic reactions or rejection. Sinus-canalis internal-fixation device 40 can also be made from a medical-grade polymer suitable for long-term or permanent internal fixation. The composition of sinus-canalis internal-fixation device 40, for example, can comprise suitable materials such as titanium, stainless steel, cobalt chrome, ceramic, high-molecular-weight polyethylene, polyetheretherketone (PEEK), polyetherketoneketone (PEKK), polymethyl-methacrylate (PMMA), polytetrafluoroethylene (PTFE), crystalline plastics, polyoxymethylene, DELRIN, and/or others.

The composition of sinus-canalis internal-fixation device 40 can also be made from a plurality of materials. For example, the composition of sinus-canalis internal-fixation device 40 can comprise a suitable blend of polymer fibers dispersed in resins such as DELRIN AF, or a suitable blend of PTFE fibers uniformly dispersed in DELRIN acetal resin.

High-grade polymers have physical properties covering the entire range of properties (such as flexibility, coefficient of friction, durability, and hardness) from metallic and plasticized materials. As a result:
a) Many compositions can be used in predetermined portions of sinus-canalis internal-fixation device 40 where the corresponding properties are most critical to structure sinus-canalis internal-fixation device 40, combining the advantageous properties of each material; or
b) The materials can be blended together in a uniform ratio throughout the entire structure of sinus-canalis internal-fixation device 40.

Post-operative imaging (fluoroscopic, magnetic resonance imaging, etc), even though not needed for proper placement of sinus-canalis internal-fixation device 40, may be desired for special purposes. In such cases, an opaque material can be added to or imbedded into sinus-canalis internal-fixation device 40.

When biotechnological techniques are applied to stimulate growth of bone cells (osteogenesis) to replace worn regions of bone, sinus-canalis internal-fixation device 40 can be made from even harder materials. Any prior-art fixation-implant material(s) can also be used to create sinus-canalis internal-fixation device 40. If desired, sinus-canalis internal-fixation device 40 can be custom-made from a three-dimensional CAD scan of the foot of a patient.

Sinus-canalis internal-fixation device 40 can vary in anatomical shape and size such that a patient will receive a precise amount of joint subluxation or dislocation correction (degree of blocking excessive exorotary motion), which is critical in special cases, such as severe deformity and incomplete sinus-canalis formation.

### Operation

Referring to prior-art figure, because prior-art implants have generally circular cross-section and sharp-edge treads integrated into their surfaces, they can not distribute the body weight of a patient like sinus-canalis internal-fixation device 40 of the present invention does. For example, because a prior-art implant 58 has generally circular cross-section and sharp-edge treads integrated into its surfaces, it can only create extremely small contact areas 59a, 59b, and 59c with talus 41 and calcaneus 42, respectively. At every step the patient makes, the whole body weight of the patient pushes in the opposite directions of arrows 60, 61, and 62, and concentrates on the extremely small contact areas 59a, 59b, and 59c at the tips of arrows 60, 61, and 62, respectively, against prior-art implant 58. As a result, prior-art implant 58 grinds, wears, deforms, and damages talus 41, calcaneus 42, surrounding tissues, ligaments, veins, arteries, and nerve systems. This leads to many problems of excruciating pain, the fracture and weakening of talus 41 and calcaneus 42, and the failure of the prior-art implantation.

In contrast, sinus-canalis internal-fixation device 40 can distribute the body weight of the patient over its entire surfaces and, thus, eliminates the above-mentioned problems of prior-art implants heretofore.

FIG. 7 illustrates the foot viewed from the front, along view direction “7” in FIG. 2. Sinus-canalis internal-fixation device 40 is shown together with the cross-sections of talus 41 and calcaneus 42. Sinus-canalis internal-fixation device 40 is properly positioned when inferior peg 45 abuts the lateral most aspect of the sinus canalis. Anatomical shaft 43 and posterior peg 46 are designed to be inserted into the expanding, lateral portion of the sinus canalis. Posterior peg 46 is in the deepest, or medial, end of the canal. The end of posterior peg 46 is shown abutting the sulcus tali. Another method, by which one ensures proper positioning, is to insert sinus-canalis internal-fixation device 40 until the end of posterior peg 46 abuts the sulcus tali. This method can be used separately or together with the method above.

As a result, anatomical shaft 43, superior peg 44, inferior peg 45, and posterior peg 46 together function to correctly position and to fixate sinus-canalis internal-fixation device 40 in the sinus canalis.

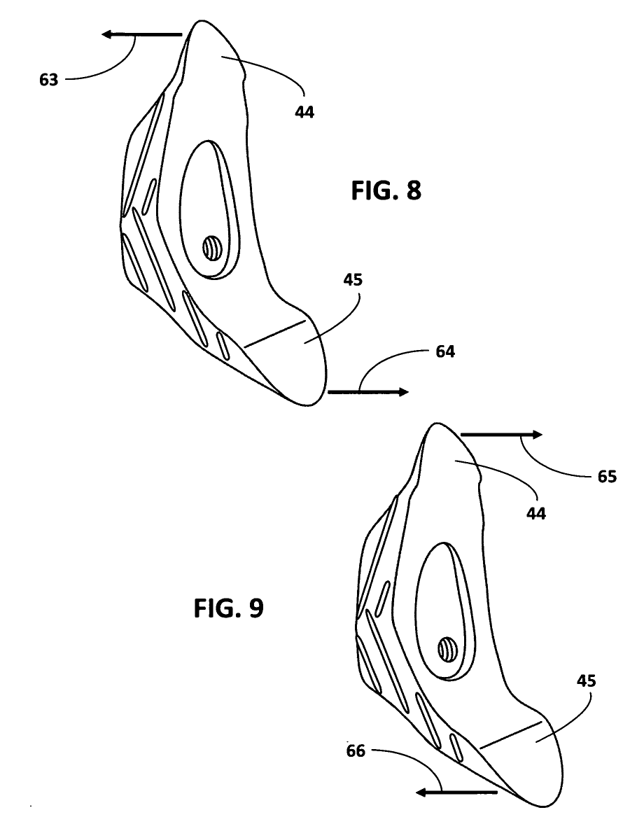

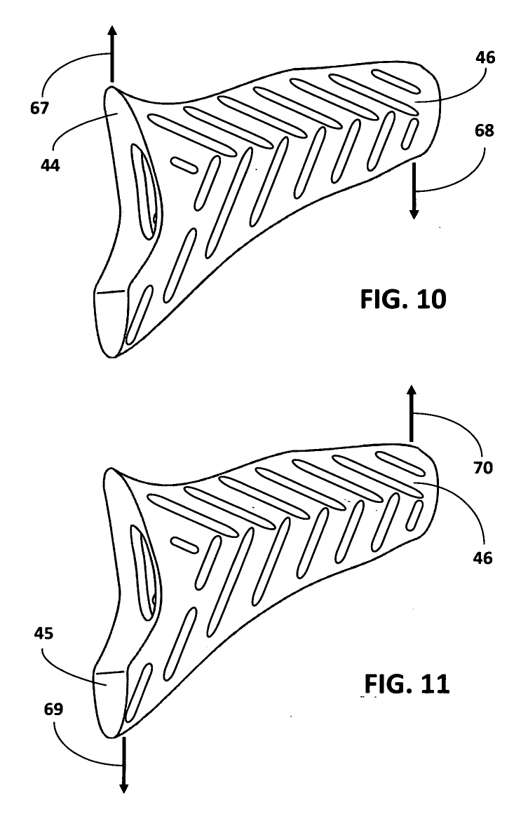

Referring to FIGS. 8, 9, 10, and 11:
a) FIG. 8 illustrates superior and inferior pegs 44 and 45. By the affect of opposite coupling forces, when superior peg 44 moves in the direction of arrow 63, inferior peg 45 counter-moves in the opposite direction of arrow 64.
FIG. 9 also illustrates superior and inferior pegs 44 and 45. By the affect of opposite coupling forces, when superior peg 44 moves in the direction of arrow 65, inferior peg 45 counter-moves in the opposite direction of arrow 66.
As a result, superior peg 44 and inferior peg 45 together function to resist the forward translation of talus 41 on calcaneus 42. This eliminates the problem of displacement that leads to failure of prior-art implants.
b) FIG. 10 illustrates superior and posterior pegs 44 and 46. By the affect of opposite coupling forces, when superior peg 44 moves in the direction of arrow 67, posterior peg 46 counter-moves in the opposite direction of arrow 68.
FIG. 11 also illustrates inferior and posterior pegs 45 and 46. By the affect of opposite coupling forces, when inferior peg 45 moves in the direction of arrow 69, posterior peg 46 counter-moves in the opposite direction of arrow 70.
As a result, superior peg 44, inferior peg 45, and posterior peg 46 together function to prevent superior and inferior dorsal togglings of sinus-canalis internal-fixation device 40 within the sinus canalis. This eliminates the problem of displacement that leads to failure of prior-art implants.

Therefore, anatomical shaft 43, superior peg 44, inferior peg 45, and posterior peg 46 together function to block anterior, medial translation and internal, medial rotation of talus 41 on calcaneus 42. Thus, sinus-canalis internal fixation device 40 blocks hyperpronation (excessive exorotation) of the foot while, at the same time, allowing normal flexion of the foot.

To properly prevent abnormal motion while allowing normal motion, sinus-canalis internal-fixation device 40 has predetermined dimensions and is capable of being custom-made to order. Sinus-canalis internal-fixation device 40 is selected to be large enough to anatomically fit the sinus canalis to prevent the collapse of the sinus canalis, but not to interfere with normal foot motion.

For example, sinus-canalis internal-fixation device 40 can be modeled and machined after a three-dimensional CAD scan of a sinus canalis of a patient.

For another example, the largest diameter of anatomical shaft 43 can range from 0.5 cm to 1.6 cm in 1 mm or 1.5 mm increment.

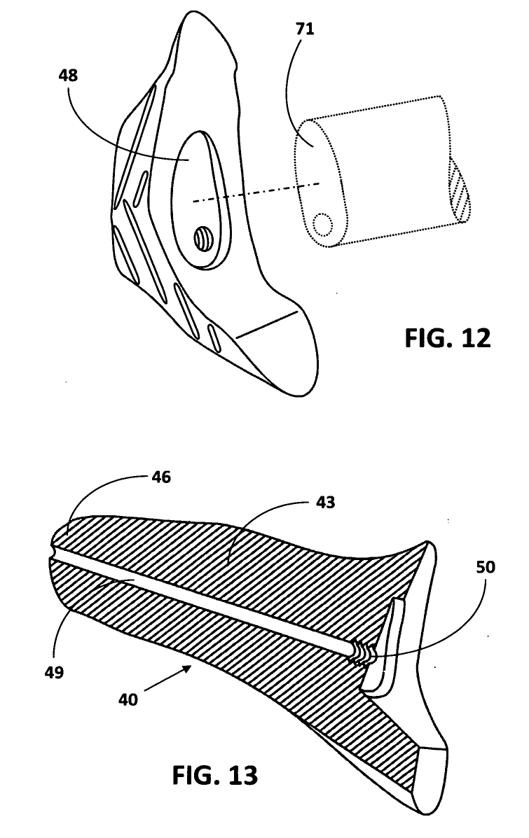

FIG. 12 illustrates insertion recess 48 of sinus-canalis internal-fixation device 40. Insertion recess 48 is for the end 71 of an insertion device, having a mating geometric shape, to be inserted thereinto to advance sinus-canalis internal-fixation device 40 into position. Insertion recess 48 can have any geometric shape, preferably a shape in which maximum torque can be applied without slippage. For examples, preferable shapes include straight slots (flat heads), cruciform (PHILLIPS heads), hexagon, polygon, POSIDRIVE, TORX, Allen-wrench shape, the like, the equivalent, other shape, and a combination of at least two of the above shapes.

FIG. 13 illustrates a cross-sectional view of sinus-canalis internal-fixation device 40. Guiding cannula 49 traverses the entire combined lengths of anatomical shaft 43 and posterior peg 46 along an eccentric longitudinal axis. Guiding cannula 49 allows sinus-canalis internal-fixation device 40 to be placed on a guide wire to facilitate proper surgical placement. Extraction thread 50 is for the end of an extraction device, having a mating thread, to be screwed thereon to extract sinus-canalis internal-fixation device 40 out of position.

As illustrated in the prior-art figure mentioned above in the operation section (See drawing sheet 4), because prior-art implants have generally circular cross-section and sharp-edge treads integrated into their surfaces, they can only create extremely small contact areas with talus 41 and calcaneus 42, respectively, and, thus, they can not distribute the body weight of a patient like sinus-canalis internal-fixation device 40 of the present invention does. At every step the patient makes, the whole body weight of the patient concentrates on the extremely small contact areas against the prior-art implant. As a result, prior-art implants grind, wear, deform, and damage talus 41, calcaneus 42, surrounding tissues, ligaments, veins, arteries, and nerve systems.

In contrast, sinus-canalis internal-fixation device 40 can distribute the body weight of the patient over its entire surfaces and, thus, can eliminate the above-mentioned problems of prior-art implants heretofore.

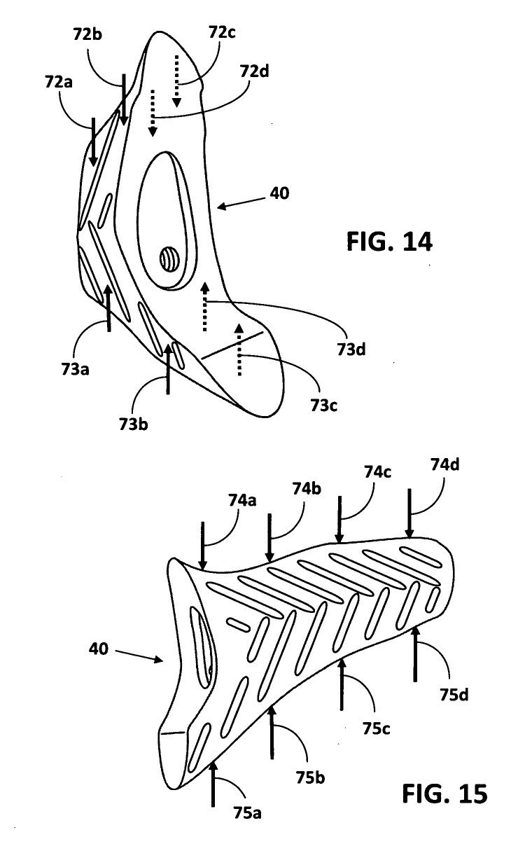

FIGS. 14 and 15 illustrate anatomical shaft 43, superior peg 44, inferior peg 45, and posterior peg 46. Anatomical shaft 43, superior peg 44, inferior peg 45, and posterior peg 46 of sinus-canalis internal-fixation device 40 of the present invention all have anatomical surfaces, which are free of prior-art extremely narrow contact surfaces, are free of prior-art sharp-edge treads, and mirror the surrounding anatomical surfaces of talus 41 and calcaneus 42. As a result, anatomical shaft 43, superior peg 44, inferior peg 45, and posterior peg 46:
a) Distribute the bodyweight of a patient over the entire surfaces of anatomical shaft 43, superior peg 44, inferior peg 45, and posterior peg 46 in the directions of arrows 72a, 72b, 72c, 72d, 73a, 73b, 73c, and 73d, respectively.
b) Distribute the bodyweight of a patient along the entire surfaces of anatomical shaft 43, superior peg 44, inferior peg 45, and posterior peg 46 in the directions of arrows 74a, 74b, 74c, 74d, 75a, 75b, 75c, and 75d, respectively.
c) Absorb shocks, caused by the bodyweight of a patient at every step the patient makes, by structuring the entire surfaces of sinus-canalis internal-fixation device 40 to mirror the anatomically irregular surfaces of talus 41 and calcaneus 42 surrounding the sinus canalis, to distribute the bodyweight of the patient over the entire surfaces of sinus-canalis internal-fixation device 40.
d) Offer unprecedented fit, by being modeled after the anatomical form and dimensions of the sinus canalis such that the entire surfaces of sinus-canalis internal-fixation device 40 mirror the entire surrounding anatomical surfaces of talus 41 and calcaneus 42;
e) Offer unprecedented comfort, by being modeled after the anatomical form and dimensions of the sinus canalis such that the entire surfaces of sinus-canalis internal-fixation device 40 mirror the entire surrounding anatomical surfaces of talus 41 and calcaneus 42, and are free of prior-art circular cross-section and sharp-edge threads.

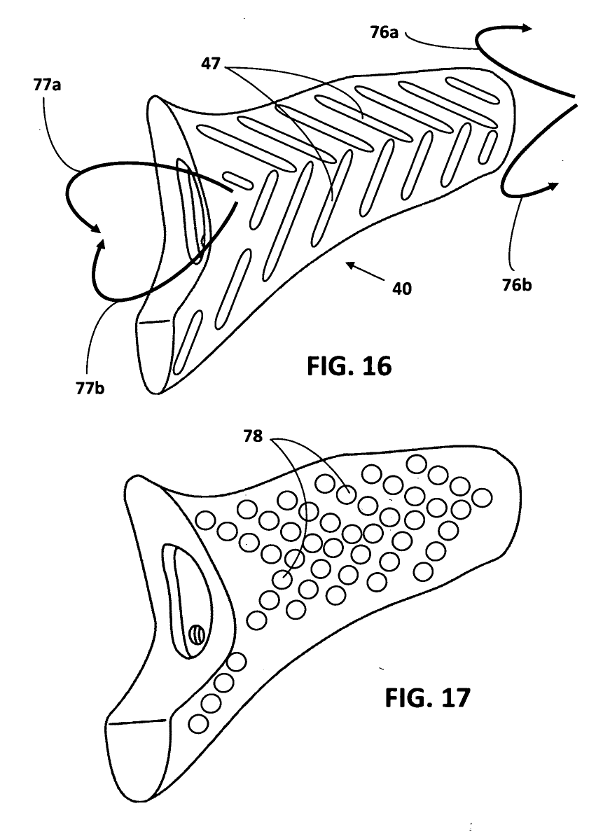

FIG. 16 illustrates a plurality of left and right forward-moving-only grooves 47. Left and right forward-moving-only grooves 47 have predetermined shapes and sizes and are disposed at predetermined locations and orientations in an arrowhead-like disposition such that left and right forward-moving-only grooves 47 point toward left and right longitudinal axes of the left and right sides of sinus-canalis internal-fixation device 40, respectively. Functioning similarly as an arrowhead, left and right forward-moving-only grooves 47: a) Push tissues outwards in the directions of arrows 76a and 76b when advancing to allow sinus-canalis internal fixation device 40 to be inserted easily into the sinus canalis; b) Push tissues inwards in the directions of arrows 77a and 77b when backing up to prevent the displacement of sinus-canalis internal-fixation device 40; and c) Secure sinus-canalis internal-fixation device 40.

As a result, this implant-securing method of the present invention overcomes the failure of prior-art implant-anchoring methods. Because the prior-art implant-anchoring methods use sharp-edge threads to cut into and thus damage talus 41, calcaneus 42, surrounding tissues, ligaments, veins, arteries, and nerve systems, or drill a vertical hole in the dorsal aspect of calcaneus 42, the prior-art implant-anchoring methods lead to many problems of excruciating pain, the fracture and weakening of talus 41 and calcaneus 42, and the failure of the prior-art implantation.

Sinus-canalis internal-fixation device 40 of the present invention provides a long-term sinus-canalis internal-fixation implant with expected useful life ranging from a period of years to a period of decades. Sinus-canalis internal-fixation device 40 of the present invention is intended to be operably a permanent sinus-canalis internal-fixation implant, one rarely or never requiring replacement over the lifetime of a patient. For example, sinus-canalis internal-fixation device 40 can be made from selected material(s), soft enough to prevent excessive wear and deformation of the surrounding bones causing undesirable side affects, but durable enough to prevent excessive wear and deformation of sinus-canalis internal-fixation device 40 causing implant failure or requiring premature replacement.

### Variations and Ramifications

Each component of sinus-canalis internal-fixation device 40 can vary in shape, size, location, and orientation.

FIG. 17 illustrates an example of sinus-canalis internal-fixation device 40. Wherein, left and right forward-moving-only grooves 47 can be replaced with left and right forward-moving-only openings or channels 78. Left and right forward-moving-only openings or channels 78 function similarly to left and right forward-moving-only grooves 47.

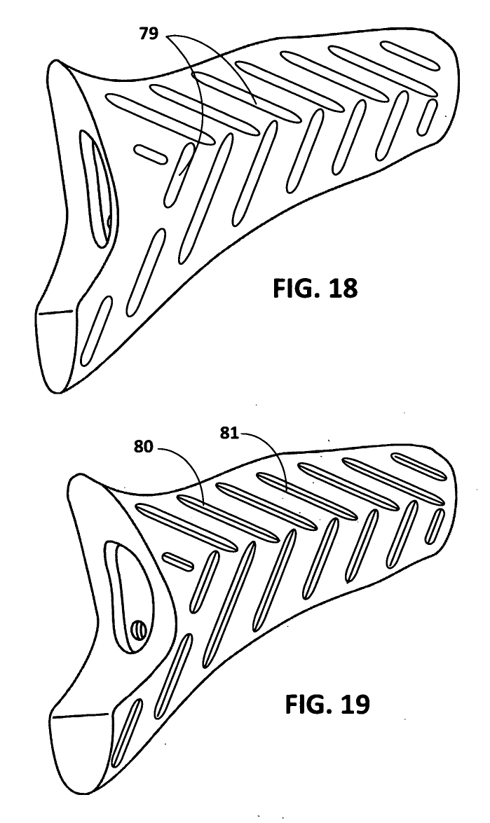

FIG. 18 illustrates an example of sinus-canalis internal-fixation device 40. Wherein, left and right forward-moving-only grooves 47 can be replaced with left and right forward-moving-only ridges 79. Left and right forward-moving-only ridges 79 function similarly to left and right forward-moving-only grooves 47.

FIG. 19 illustrates an example of sinus-canalis internal-fixation device 40. Wherein, left and right forward-moving-only grooves 47 can be replaced with left and right forward-moving-only ridges 80 and left and right forward-moving-only grooves 81. Left and right forward-moving-only ridges 80 and left and right forward-moving-only grooves 81 function similarly to left and right forward-moving-only grooves 47.

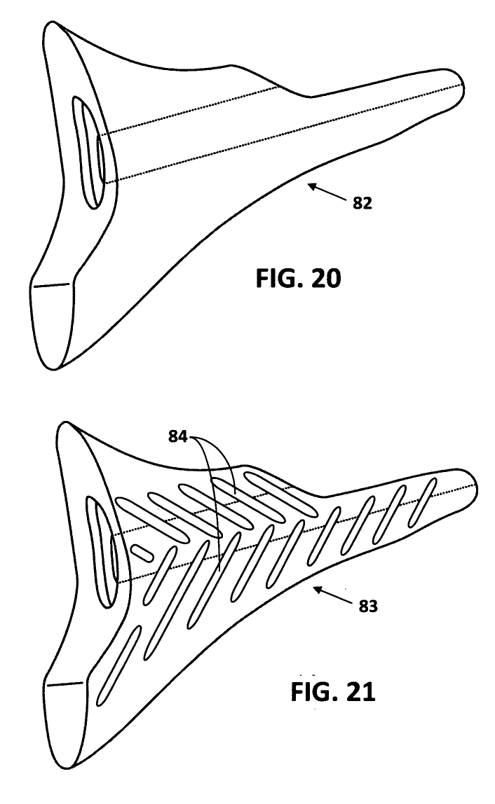

FIG. 20 illustrates a sinus-canalis internal-fixation device 82. Sinus-canalis internal-fixation device 82 is equivalent to and functions similarly to sinus-canalis internal-fixation device 40. Sinus-canalis internal-fixation device 82 comprises sinus-canalis internal-fixation device 40 with anatomical shaft 43 and posterior peg 46 having different shapes, respectively, to create predetermined space(s) for selected tissues, ligaments, veins, arteries, and/or nerve systems.

FIG. 21 illustrates a sinus-canalis internal-fixation device 83. Sinus-canalis internal-fixation device 83 is equivalent to and functions similarly to sinus-canalis internal-fixation device 40. Sinus-canalis internal-fixation device 83 comprises sinus-canalis internal-fixation device 82 in FIG. 20 above, which has left and right forward-moving-only grooves 84 integrated thereinto at predetermined locations and orientations. Left and right forward-moving-only grooves 84 are equivalent to and function similarly to left and right forward-moving-only grooves 47.

Sinus-canalis internal-fixation device 40 can have various tissue-engagement surfaces to promote interactions with surrounding connective tissues and ligaments within the sinus canalis. For example, the tissue-engagement surfaces of sinus-canalis internal-fixation device 40 can have at least one recess, opening, ridge, hill, the like, the equivalent, or a combination of at least two of the above (e.g., groove, channel, canal, hole, through-hole, pore, micropore, etc.) to allow fibrous-tissue ingrowth to operably engage the surrounding connective tissues and ligaments. As a result, this firmly and permanently anchors sinus-canalis internal fixation device 40 in place.

The cross-section of any portion of sinus-canalis internal-fixation device 40 can have any shape. For example, the cross-section of a portion of sinus-canalis internal-fixation device 40 can be ovoid, elliptical, the like, etc.

### Surgical Procedure

For example, the sinus-canalis internal-fixation instrumentation can include: a guide, a cannulated incising device, a set of cannulated sizing devices, a set of sinus-canalis internal-fixation devices 40, and a cannulated inserting device.

Each of sinus-canalis internal-fixation devices 40 can increase, for example, 1 mm in diameter from 0.5 cm to 1.6 cm.

Each of the cannulated sizing devices can increase, for example, 1 mm or 1.5 mm in diameter from 0.5 cm to 1.6 cm.

To perform a sinus-canalis internal-fixation surgery:

First, a 1-cm-to-2-cm incision is made and deepened into the sinus canalis of a foot.

Next, the guide (e.g., a guide wire or a guide pin) is inserted into the sinus canalis and is left in place until the end of the procedure. The angle of the guide is dictated by the anatomical angle of the sinus canalis.

Next, the incising device is inserted over the guide into the sinus canalis to selectively transect the interosseous ligament.

Next, the smallest-diameter sizing device is inserted over the guide into the sinus canalis.

Next, the smallest-diameter sizing device is replaced with a subsequent larger-diameter sizing device until the appropriate size is determined.

Next, the sizing device of the appropriate size is removed.

Next, one sinus-canalis internal-fixation device 40 of the appropriate size is inserted over the guide.

Next, the inserting device is inserted over the guide and into insertion recess 48 of sinus-canalis internal-fixation device 40.

Next, through the action of the inserting device (which, for example, functions like an alien wrench), sinus-canalis internal-fixation device 40 is advanced into the sinus canalis until proper placement of sinus-canalis internal-fixation device 40 is achieved. Proper placement of sinus-canalis internal-fixation device 40 is achieved when superior and inferior pegs 44 and 45 of sinus-canalis internal-fixation device 40 abut the lateral most aspect of the sinus canalis (See FIG. 7).

If desired, sinus-canalis internal-fixation device 40 can be oscillated into position by use of any conventional method of applying torque, including the use of manual and power devices.

After sinus-canalis internal-fixation device 40 is fully inserted, and the guide and inserting device are removed, the incision is closed. The method of closure of the incision is a surgeon's choice.

### Conclusion

Any component of sinus-canalis internal-fixation device 40 can have any shape and size. The cross-section of any portion of any component of sinus-canalis internal-fixation device 40 can have any shape and size. Any component of sinus-canalis internal-fixation device 40 can curve in any direction in respect to its longitudinal axis. Any component of sinus-canalis internal-fixation device 40 can twist in any direction in respect to its longitudinal axis. Each component of sinus-canalis internal-fixation device 40 can anatomically be modeled after a corresponding portion of a sinus canalis of any patient.

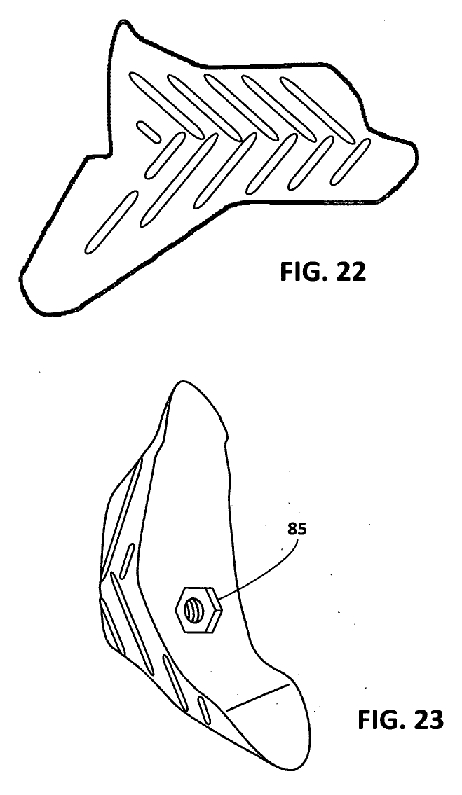

For example, FIG. 22 illustrates the side view of a sinus-canalis internal-fixation device, which is equivalent to and functions similarly to sinus-canalis internal-fixation device 82 illustrated in FIG. 20 above. The insertion recess of this sinus-canalis internal-fixation device has a hexagonal shape, which is equivalent to and functions similarly to the insertion recess of sinus-canalis internal-fixation device 82. Equivalent to sinus-canalis internal-fixation device 82, this sinus-canalis internal-fixation device has a predetermined shape to create predetermined space(s) for selected tissues, ligaments, veins, arteries, and/or nerve systems. Further, if desired, left and right grooves, ridges, recesses, openings, channels, or the like are integrated into the left and right sides of this sinus-canalis internal-fixation device, respectively, at predetermined locations and orientations. The left and right grooves, ridges, recesses, openings, channels, or the like are equivalent to and function similarly to left and right forward-moving-only grooves 47.

For another example, FIG. 23 illustrates the front view of a sinus-canalis internal-fixation device, which is equivalent to and functions similarly to sinus-canalis internal-fixation device 82 illustrated in FIG. 20 above. This sinus-canalis internal-fixation device has an insertion recess 85 of a hexagonal shape, which is equivalent to and functions similarly to the insertion recess of sinus-canalis internal-fixation device 82.

### Important Advantages

The present invention substantially departs from the conventional concepts and designs of the prior art. In doing so, the present invention provides sinus-canalis internal-fixation device 40 having many unique and significant features (anatomical shaft 43, anatomical superior peg 44, anatomical inferior peg 45, and anatomical posterior peg 46) and advantages, which overcome all the disadvantages of the prior art, as follows:
1) One object of the invention is that sinus-canalis internal-fixation device 40 distributes the bodyweight of a patient over its entire surfaces, by being modeled after the anatomical form and dimensions of a sinus canalis, which anatomically twists and curves and is surrounded by the anatomically irregular surfaces of talus 41 and calcaneus 42.
2) Another object of the invention is that sinus-canalis internal-fixation device 40 absorbs shocks, caused by the bodyweight of a patient at every step the patient makes, by structuring its entire surfaces to mirror the anatomically irregular surfaces of talus 41 and calcaneus 42 surrounding sinus-canalis internal-fixation device 40 such that sinus-canalis internal-fixation device 40 distributes the bodyweight of the patient over its entire surfaces.
3) Another object of the invention is that sinus-canalis internal-fixation device 40 offers unprecedented fit, by being modeled after the anatomical form and dimensions of a sinus canalis such that the entire surfaces of sinus-canalis internal-fixation device 40 mirror the entire surrounding surfaces of talus 41 and calcaneus 42.
4) Another object of the invention is that sinus-canalis internal-fixation device 40 offers unprecedented comfort, by being modeled after the anatomical form and dimensions of a sinus canalis such that the entire surfaces of sinus-canalis internal-fixation device 40 mirror the entire surrounding surfaces of talus 41 and calcaneus 42, and are free of prior-art circular cross-section and sharp-edge threads.
5) Another object of the invention is that sinus-canalis internal-fixation device 40 utilizes its opposite-coupling-force blocking pegs 44, 45, and 46 to block excessive, exorotational end-range-of-motion (unraveling) of the subtalar joint and to block abnormal subluxation or dislocation between talus 41 and calcaneus 42 while maintaining normal motion and alignment.
6) Another object of the invention is that left and right forward-moving-only grooves 47 of sinus-canalis internal-fixation device 40 function similarly as an arrowhead: a) Pushing tissues outwards when advancing to allow sinus-canalis internal-fixation device 40 to be inserted easily into the sinus canalis; b) Pushing tissues inwards when backing up to prevent the displacement of sinus-canalis internal-fixation device 40; and c) Securing sinus-canalis internal-fixation device 40.
7) Another object of the invention is to obviate limitations in correcting abnormal foot mechanics.
8) Another object of the invention is to ensure proper foot motion, by stabilizing the end-range-of-motion between talus 41 and calcaneus 42.
9) Another object of the invention is to ensure that both the medial and lateral aspects of talus 41 and calcaneus 42 are stabilized.
10) A further object of this invention is to correct poor-alignment, both proximally and distally, of the joints surrounding talus 41 and calcaneus 42.
11) A further object of the invention is to provide sinus-canalis internal-fixation device 40, that will not wear or deform talus 41 and calcaneus 42 over time.
12) A further object of the invention is to provide sinus-canalis internal-fixation device 40, that will not wear or deform over time and, thus, fail.
13) A further object of the invention is to provide sinus-canalis internal-fixation device 40, that will remain in place without a separate implant-anchoring procedure.
14) Another further object of the invention is to provide a method of correctly positioning sinus-canalis internal-fixation device 40 in the sinus canalis between talus 41 and calcaneus 42 without having to verify the correct position with a fluoroscope and, thus, without exposing a patient to radiation.
15) Another further object of the invention is to provide a minimally invasive method for implanting sinus-canalis internal-fixation device 40.
16) Another further object of the invention is to provide sinus-canalis internal-fixation device 40 without requiring post-operative casting of the extremity.
17) Another further object of the invention is to provide sinus-canalis internal-fixation device 40, which allows early post-operative ambulation.

### Unique Features and Functions

Referring to FIGS. 3, 4, 5, 6, 7, 8, 9, 10, 11, 12, 13, 14, and 15, having a shape modeled after the anatomical form and dimensions of the sinus tarsi of the patient and having anatomical superior peg 44, anatomical inferior peg 45, and anatomical posterior peg 46, sinus-canalis internal-fixation device 40 can:
a) Block the anterior, medial translation and internal, medial rotation of talus 41 on calcaneus 42 of the ankle-bone structure of the patient to obviate limitations in correcting abnormal foot mechanics,
b) Distribute the body weight of the patient over a maximum contact area between sinus-canalis internal-fixation device 40, talus 41, and calcaneus 42,
c) Absorb the shocks caused by the body weight of the patient,
d) Create coupling-force affect to prevent superior and inferior togglings of sinus-canalis internal-fixation device 40 within the sinus tarsi to eliminates the problem of displacement and failure of sinus-canalis internal-fixation device 40,
e) Correct an anatomically deformed alignment of the ankle-bone structure,
f) Maintain the ankle-bone structure in an anatomically correct alignment, and
g) Eliminate the need for having to verify the anatomically correct alignment of the ankle-bone structure with a fluoroscope, and thus eliminate the need for exposing the patient to radiation.

What is claimed is:

1. An internal-fixation system for blocking anterior, medial translation and internal, medial rotation of a talus on a calcaneus of an ankle-bone structure of a patient to obviate limitations in correcting abnormal foot mechanics, for distributing body weight of the patient over a maximum contact area on the internal-fixation system, for absorbing shocks caused by the body weight of the patient, for creating coupling-force affect to prevent superior and inferior togglings of the internal-fixation system within a sinus tarsi of the ankle-bone structure to eliminates the problem of displacement and failure of the internal-fixation system, for correcting an anatomically deformed alignment of the ankle-bone structure, for maintaining the ankle-bone structure in an anatomically correct alignment, and for eliminating the need for having to verify the anatomically correct alignment of the ankle-bone structure with a fluoroscope and thus eliminating the need for exposing the patient to radiation, the internal-fixation system comprising:
an elongated body, said elongated body having a top, a bottom, a front end, and a back end, said elongated body for being inserted into a canalis-tarsi area of the patient, said elongated body generally having an anatomical shape of an elliptical cylinder, said elongated body curving sideways with respect to its longitudinal axis, said elongated body twisting with respect to its longitudinal axis, said elongated body tapering with respect to its longitudinal axis;
a first member, said first member integrated into said top of said elongated body, said first member for being inserted into a sinus area of the patient, said first member generally having an anatomical shape of a pyramid, said first member curving sideways with respect to said elongated body, said first member twisting with respect to said elongated body, said first member curving upwards with respect to said elongated body;
a second member, said second member integrated into said bottom of said elongated body, said second member for being inserted into a sinus area of the patient, said second member generally having an anatomical shape of a partially elliptical cylinder, said second member curving sideways with respect to said elongated body, said second member twisting with respect to said elongated body, said second member curving downwards with respect to said elongated body;
a third member, said third member integrated into said back end of said elongated body, said third member for being inserted into a canalis-tarsi area of the patient, said third member generally having an anatomical shape of a partial cone, said third member curving downwards with respect to said elongated body;
a recess, said recess generally having a hexagonal shape, said recess integrated into said front end of said elongated body for an insertion means to be inserted therein to advance the internal-fixation system into the sinus tarsi of the patient; and
a bore, said bore generally having a round or elliptical cross-section, said bore extending the combined length of said elongated body and said third member for allowing placement of the internal-fixation system on a guide to facilitate accurate surgical implantation, said bore having a bore end adjacent to said front end of said elongated body, said bore end being threaded for an extraction means to be screwed therein to extract the internal-fixation system out of the sinus tarsi of the patient,
Whereby, provided is the internal-fixation system, which is generally modeled after the anatomical form and dimensions of the sinus tarsi of the patient, blocks the anterior, medial translation and internal, medial rotation of the talus on the calcaneus of the ankle-bone structure of the patient to obviate limitations in correcting abnormal foot mechanics, distributes the body weight of the patient over a maximum contact area on the internal-fixation system, absorbs the shocks caused by the body weight of the patient, creates coupling-force affect to prevent superior and inferior togglings of the internal-fixation system within the the sinus tarsi to eliminates the problem of displacement and failure of the internal-fixation system, corrects an anatomically deformed alignment of the ankle-bone structure, maintains the ankle-bone structure in an anatomically correct alignment, and eliminates the need for having to verify the anatomically correct alignment of the ankle-bone structure with a fluoroscope and thus eliminates the need for exposing the patient to radiation.

2. The internal-fixation system of claim 1, wherein having opposite sides, the internal-fixation system further comprising a plurality of predetermined grooves integrated into said opposite sides for pushing tissues of the patient away from or toward the internal-fixation system when the internal-fixation system advances into or backs out of the sinus tarsi respectively, and for stimulating and permitting tissue ingrowth to anchor the internal-fixation device inside the sinus tarsi.

3. The internal-fixation system of claim 1, wherein having opposite sides, the internal-fixation system further comprising a plurality of predetermined elements integrated into said opposite sides, said predetermined elements selected from the group consisting of: ridges, nipples, recesses, openings, channels, and a combination of at least two of the above.

4. The internal-fixation system of claim 1, wherein said bore end being threaded cylindrically or conically.

5. The internal-fixation system of claim 1, wherein at least one element of the internal-fixation system made of a material selected from the group consisting of:
titanium, stainless steel, cobalt chrome, ceramic, high-molecular-weight polyethylene, polyetheretherketone (PEEK), polyetherketoneketone (PEKK), polymethyl-methacrylate (PMMA), polytetrafluoroethylene (PTFE), crystalline plastics, polyoxymethylene, DELRIN, and a combination of at least two of the above.

6. The internal-fixation system of claim 1, wherein the outer diameter of said elongated body ranging from 0.5 cm to 1.6 cm.

7. The internal-fixation system of claim 1, wherein a section of said bore extending along the edge of the internal-fixation system such that the inside of said section is exposed.

8. An internal-fixation system for blocking anterior, medial translation and internal, medial rotation of a talus on a calcaneus of an ankle-bone structure of a patient to obviate limitations in correcting abnormal foot mechanics, for distributing body weight of the patient over a maximum contact area on the internal-fixation system, for absorbing shocks caused by the body weight of the patient, for creating coupling-force affect to prevent superior and inferior togglings of the internal-fixation system within a sinus tarsi of the ankle-bone structure to eliminates the problem of displacement and failure of the internal-fixation system, for correcting an anatomically deformed alignment of the ankle-bone structure, for maintaining the ankle-bone structure in an anatomically correct alignment, and for eliminating the need for having to verify the anatomically correct alignment of the ankle-bone structure with a fluoroscope and thus eliminating the need for exposing the patient to radiation, the internal-fixation system comprising:
an elongated body, said elongated body having a top, a bottom, a front end, and a back end, said elongated body for being inserted into a canalis-tarsi area of the patient, said elongated body having a shape generally modeled after a corresponding portion of the sinus tarsi of the patient;
a first member, said first member integrated into said top of said elongated body, said first member for being inserted into a sinus area of the patient, said first member having a shape generally modeled after a corresponding portion of the sinus tarsi of the patient;
a second member, said second member integrated into said bottom of said elongated body, said second member for being inserted into a sinus area of the patient, said second member having a shape generally modeled after a corresponding portion of the sinus tarsi of the patient; and
a third member, said third member integrated into said back end of said elongated body, said third member for being inserted into a canalis-tarsi area of the patient, said third member having a shape generally modeled after a corresponding portion of the sinus tarsi of the patient,
Whereby, provided is the internal-fixation system, which blocks the anterior, medial translation and internal, medial rotation of the talus on the calcaneus of the ankle-bone structure of the patient to obviate limitations in correcting abnormal foot mechanics, distributes the body weight of the patient over a maximum contact area on the internal-fixation system, absorbs the shocks caused by the body weight of the patient, creates coupling-force affect to prevent superior and inferior togglings of the internal-fixation system within the sinus tarsi to eliminates the problem of displacement and failure of the internal-fixation system, corrects an anatomically deformed alignment of the ankle-bone structure, maintains the ankle-bone structure in an anatomically correct alignment, and eliminates the need for having to verify the anatomically correct alignment of the ankle-bone structure with a fluoroscope and thus eliminates the need for exposing the patient to radiation.

9. The internal-fixation system of claim 8, wherein having opposite sides, the internal-fixation system further comprising a plurality of predetermined grooves integrated into said opposite sides for pushing tissues of the patient away from or toward the internal-fixation system when the internal-fixation system advances into or backs out of the sinus tarsi respectively, and for stimulating and permitting tissue ingrowth to anchor the internal-fixation device inside the sinus tarsi.

10. The internal-fixation system of claim 8, wherein having opposite sides, the internal-fixation system further comprising a plurality of predetermined elements integrated into said opposite sides, said predetermined elements selected from the group consisting of: ridges, nipples, recesses, openings, channels, and a combination of at least two of the above.

11. The internal-fixation system of claim 8, wherein the anatomical shape of the sinus tarsi of the patient CAD-scanned for the internal-fixation system to be generally modeled after.

12. The internal-fixation system of claim 8, further comprising a predetermined recess and a predetermined bore, said recess integrated into said front end of said elongated body for an insertion means to be inserted therein to advance the internal-fixation system into the sinus tarsi of the patient, said bore extending the combined length of said elongated body and said third member for allowing placement of the internal-fixation system on a guide to facilitate accurate surgical implantation, said bore having a bore end adjacent to said front end of said elongated body, said bore end being threaded for an extraction means to be screwed therein to extract the internal-fixation system out of the sinus tarsi of the patient.

13. The internal-fixation system of claim 12, wherein said recess having a hexagonal shape.

14. The internal-fixation system of claim 12, wherein said recess having a polygonal shape.

15. The internal-fixation system of claim 12, wherein said bore end being threaded cylindrically or conically.

16. The internal-fixation system of claim 12, wherein a section of said bore extending along the edge of the internal-fixation system such that the inside of said section is exposed.

17. The internal-fixation system of claim 8, wherein at least one element of the internal-fixation system made of a material selected from the group consisting of: titanium, stainless steel, cobalt chrome, ceramic, high-molecular-weight polyethylene, polyetheretherketone (PEEK), polyetherketoneketone (PEKK), polymethyl-methacrylate (PMMA), polytetrafluoroethylene (PTFE), crystalline plastics, polyoxymethylene, DELRIN, and a combination of at least two of the above.

18. The internal-fixation system of claim 8, wherein the outer diameter of said elongated body ranging from 0.5 cm to 1.6 cm.

19. A method for blocking anterior, medial translation and internal, medial rotation of a talus on a calcaneus of an ankle-bone structure of a patient to obviate limitations in correcting abnormal foot mechanics, for distributing body weight of the patient over a maximum contact area on an internal-fixation system, for absorbing shocks caused by the body weight of the patient, for creating coupling-force affect to prevent superior and inferior togglings of the internal-fixation system within a sinus tarsi of the ankle-bone structure to eliminates the problem of displacement and failure of the internal-fixation system, for preventing superior and inferior dorsal togglings of the internal-fixation system, for correcting an anatomically deformed alignment of the ankle-bone structure, for maintaining the ankle-bone structure in an anatomically correct alignment, and for eliminating the need for having to verify the anatomically correct alignment of the ankle-bone structure with a fluoroscope and thus eliminating the need for exposing the patient to radiation, providing the internal-fixation system, the internal-fixation system comprising:
an elongated body, said elongated body having a top, a bottom, a front end, and a back end, said elongated body for being inserted into a canalis-tarsi area of the patient, said elongated body having a shape generally modeled after a corresponding portion of the sinus tarsi of the patient;
a first member, said first member integrated into said top of said elongated body, said first member for being inserted into a sinus area of the patient, said first member having a shape generally modeled after a corresponding portion of the sinus tarsi of the patient;
a second member, said second member integrated into said bottom of said elongated body, said second member for being inserted into a sinus area of the patient, said second member having a shape generally modeled after a corresponding portion of the sinus tarsi of the patient; and
a third member, said third member integrated into said back end of said elongated body, said third member for being inserted into a canalis-tarsi area of the patient, said third member having a shape generally modeled after a corresponding portion of the sinus tarsi of the patient,
the method comprising the step of implanting the internal-fixation system in the sinus tarsi of the patient,
whereby, the internal-fixation system blocks the anterior, medial translation and internal, medial rotation of the talus on the calcaneus of the ankle-bone structure of the patient to obviate limitations in correcting abnormal foot mechanics, distributes the body weight of the patient over a maximum contact area on the internal-fixation system, absorbs the shocks caused by the body weight of the patient, creates coupling-force affect to prevent superior and inferior togglings of the internal-fixation system within the the sinus tarsi to eliminates the problem of displacement and failure of the internal-fixation system, corrects an anatomically deformed alignment of the ankle-bone structure, maintains the ankle-bone structure in an anatomically correct alignment, and eliminates the need for having to verify the anatomically correct alignment of the ankle-bone structure with a fluoroscope and thus eliminates the need for exposing the patient to radiation.

20. The method of claim 19, wherein the internal-fixation system further comprising a predetermined recess and a predetermined bore, said recess integrated into said front end of said elongated body for an insertion means to be inserted therein to advance the internal-fixation system into the sinus tarsi of the patient, said bore extending the combined length of said elongated body and said third member for allowing placement of the internal-fixation system on a guide to facilitate accurate surgical implantation, said bore having a bore end adjacent to said front end of said elongated body, said bore end being threaded for an extraction means to be screwed therein to extract the internal-fixation system out of the sinus tarsi of the patient.
